(19) United States
(12) Patent Application Publication
(10) Pub. No.: US 2011/0091964 A1
(43) Pub. Date: Apr. 21, 2011

(54) CELL MANIPULATION OBSERVATION APPARATUS

(75) Inventor: Kiyohiko TATEYAMA, Machida-shi (JP)

(73) Assignee: OLYMPUS CORPORATION, Tokyo (JP)

(21) Appl. No.: 12/977,232

(22) Filed: Dec. 23, 2010

Related U.S. Application Data
(63) Continuation of application No. PCT/JP2009/060958, filed on Jun. 16, 2009.

(30) Foreign Application Priority Data
Jun. 27, 2008 (JP) 2008-168067

(51) Int. Cl. C12M 1/34; C12M 1/00

(52) U.S. Cl. 435/287.1; 435/283.1

(57) ABSTRACT

An introducing position arrangement unit arranges a position of a cell into which a substance is introduced, of the cells in a culture dish mounted on a stage, specified by a specification unit to a predetermined cell manipulation position in a viewing field of an observation unit by stage driving. A cell manipulation unit introduces an introduction substance mixed in a culture fluid in the culture dish into the cell by making a hole on a cell membrane of the cell moved to the predetermined cell manipulation position. A diagram display unit displays a diagram associated with an observation range which is a range of the culture dish observable by the observation unit and also displays a cell manipulation range which is a cell manipulation enabled range and a position currently observed by the observation unit to be superimposed on the diagram associated with the observation range.

### CROSS REFERENCE TO RELATED APPLICATIONS

This is a Continuation Application of PCT Application No. PCT/JP2009/060958, filed Jun. 16, 2009, which was published under PCT Article 21(2) in Japanese.

This application is based upon and claims the benefit of priority from prior Japanese Patent Application No. 2008-168067, filed Jun. 27, 2008, the entire contents of which are incorporated herein by reference.

### BACKGROUND OF THE INVENTION

1. Field of the Invention

The present invention relates to a cell manipulation observation apparatus that introduces a substance such as a gene into a cell to observe the cell having the substance introduced therein.

2. Description of the Related Art

Jpn. Pat. Appln. KOKAI Pub. No. JP-A 2005-326341 (Patent Document 1) discloses a cell position teaching method and a single-cell manipulation assistance robot capable of reproducing a position of an introduced cell by selecting a cell to be manipulated in a microscopic field, recording a coordinate value of the selected cell, providing two reference points, and performing coordinate transformation even if a culture dish is rearranged. As a result, a cell in a microscopic field can be arbitrarily selected while viewing a display on a monitor, thereby carrying out the cell manipulation.

Further, Jpn. Pat. Appln. KOKAI Pub. No. JP-A 2006-141326 (Patent Document 2) discloses a culture apparatus that displays a diagram associated with a culture dish and also displays a current camera position, a position to be moved, and a record of observed positions in the past. As a result, an operator can confirm a position in the culture dish which is being observed based on the display on the monitor.

In general, a culture dish has a large region with respect to a microscopic field range, and its periphery is bounded by a sidewall in order to immerse cells in a liquid solution. Therefore, when a cell manipulation tool configured to manipulate a cell or a microscope stage on which the culture dish is mounted is unintentionally moved, the cell manipulation tool and the culture dish interfere with each other, and the cell manipulation tool strikes the cell in the culture dish, thus damaging the cell in some cases.

Therefore, according to the technology in Patent Document 1, since the microscopic field of just a small part in the culture dish is displayed, it is difficult for the operator to ascertain a position in the culture dish at which the cell manipulation is carried out, and the cell manipulation tool may possibly strike the cell.

Furthermore, when performing the cell manipulation, a range in which the manipulation can be safely performed without interference differs depending on a shape of the cell manipulation tool and a shape of the culture dish. Therefore, when a diagram associated with the culture dish like that according to the technology in Patent Document 2 alone is displayed, the range in which the manipulation can be safely effected is hard to ascertain, and the cell manipulation tool may still possibly strike the cell.

### BRIEF SUMMARY OF THE INVENTION

In view of the above-described problem, it is an object of the present invention to provide a cell manipulation observation apparatus that can eliminate the possibility that the cell manipulation tool is unintentionally caused to strike the cell.

According to an aspect of the present invention, there is provided a cell manipulation observation apparatus which introduces a substance into a cell and observes the cell, comprising:

a stage configured to mount a culture dish and move the culture dish to horizontal;

an observation unit configured to acquire and display an image of the cells in the culture dish mounted on the stage;

a diagram display unit configured to display a diagram associated with an observation range which is a range of the culture dish observable by the observation unit and also display a cell manipulation range which is a cell manipulation enabled range and a position currently observed by the observation unit to be superimposed on the diagram associated with the observation range;

a specification unit configured to specify a position of a cell into which a substance is introduced of the cells in the culture dish mounted on the stage;

an introduction position arrangement unit configured to arrange the position specified by the specification unit to a predetermined cell manipulation position within a viewing field of the observation unit by driving the stage; and

a cell manipulation unit having a cell manipulation tool and configured to introduce an introduction substance mixed in a culture fluid in the culture dish into the cell by using the cell manipulation tool to perforate a cell membrane of the cell which has been moved to the predetermined cell manipulation position by the introduction position arrangement unit.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is an overall structural view showing a cell manipulation observation apparatus according to a first embodiment of the present invention;

FIG. 2 is a view showing a configuration concerning attachment of an adapter of a tip drive device;

FIG. 3 is a view showing a configuration of a needle;

FIG. 4 is a view for explaining interference caused by angles of the needle;

FIG. 5 is a view for explaining a movable range;

FIG. 6 is a view showing a top view and a cross-sectional view of a glass base dish;

FIG. 7 is a view for explaining a movable range in case of the glass base dish;

FIG. 8 is a view showing a flowchart for explaining a flow of cell manipulation and an observation method using the cell manipulation observation apparatus according to the first embodiment;

FIG. 9A is a view showing a first half part of a series of flowcharts for explaining a control program for a cell manipulation/observation operation executed by a control computer;

FIG. 9B is a view showing a last half part of the series of flowcharts for explaining the control program for the cell manipulation/observation operation executed by the control computer;

FIG. 10 is a view showing an example of an operation window displayed on a monitor;

FIG. 11 is a view showing a display example of a monitor display portion when specifying a position of a tip portion;

FIG. 12 is a view showing a flowchart of a first mode cell introducing operation and a second mode cell introducing operation; and

FIG. 13 is a view showing a flowchart of an XY-stage movement command operation in a cell manipulation observation apparatus according to a second embodiment of the present invention.

### DETAILED DESCRIPTION OF THE INVENTION

The best mode for carrying out the present invention will now be described hereinafter with reference to the drawings.

### First Embodiment

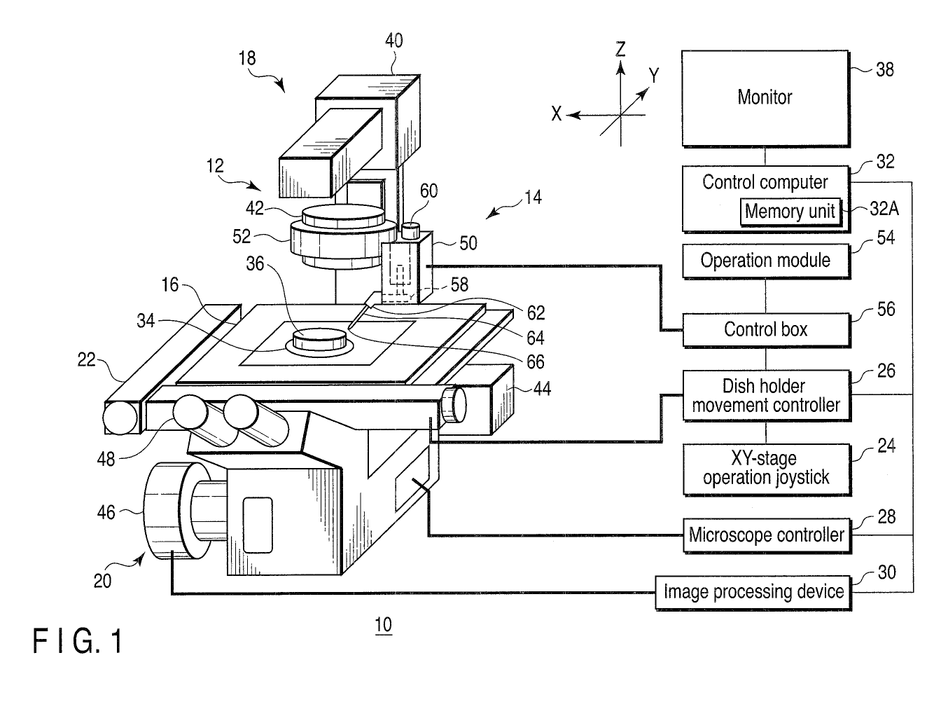

A cell manipulation observation apparatus 10 according to a first embodiment of the present invention includes an inverted microscope 12 configured to observe cells and a tip drive device 14 disposed to this microscope as shown in FIG. 1.

The inverted microscope 12 includes a dish holder 16, an illumination device 18, an observation device 20, a microscope XY-stage 22, an XY-stage operation joystick 24, a dish holder movement controller 26, a microscope controller 28, an image processing device 30, and a control computer 32.

The dish holder 16 includes a θ table 34 on which a dish 36 is mounted, the dish 36 being a culture dish in which cells are cultured. The illumination device 18 illuminates cells in the dish holder 16. The observation device 20 is utilized to observe light reflected from or transmitted through the cell or fluorescent light emanating from the cell. The microscope XY-stage 22 moves the dish holder 16 in an X-direction and a Y-direction. The XY-stage operation joystick 24 is an operation member that is used by an operator to instruct movement of the microscope XY-stage 22. The dish holder movement controller 26 drives and controls the microscope XY-stage 22 and the θ table 34 in depending on a command from the XY-stage operation joystick 24. The microscope controller 28 controls the illumination device 18 and the observation device 20. The image processing device 30 processes an image obtained by the observation device 20. The control computer 32 controls the entire inverted microscope 12 through the dish holder movement controller 26 and the microscope controller 28. To the control computer 32 is connected a monitor 38 configured to display an image processed by the image processing device 30. Therefore, the observation device 20, the image processing device 30, the control computer 32, and the monitor 38 function as an observation unit configured to acquire and display an image of cells in the dish holder 16. Further, the control computer 32 includes a memory unit 32A configured to store various kinds of information.

Furthermore, the illumination device 18 includes a transmitted light source 40, a condenser lens 42, and a reflected light source 44. The transmitted light source 40 irradiates cells with illumination light from an opposite side of the observation device 20. The condenser lens 42 condenses the illumination light emitted from the transmitted light source 40 onto the cells. The reflected light source 44 irradiates the cells with illumination light from the same direction as the observation device 20.

On the other hand, the observation device 20 includes a non-illustrated observation optical system including an objective lens, a CCD camera 46, and an eyepiece lens 48. The CCD camera 46 acquires light from the cells through the observation optical system to obtain an image. The eyepiece lens 48 is utilized by the operator to directly observe light from the cells.

Moreover, the tip drive device 14 according to this embodiment is composed of a main unit 50, a microscope adapter 52, an operation module 54, and a control box 56. The microscope adapter 52 is an attachment portion of the main unit 50 for the condenser lens 42. The operation module 54 can be set at any desired position. The control box 56 controls the main unit 50 in accordance with an operation of the operation module 54 by the operator. This control box 56 is connected to the control computer 32 through the dish holder movement controller 26 and configured to enable control over the main unit 50 by using the control computer 32. Additionally, FIG. 1 shows a state that the main unit 50 is attached at the right side of the condenser lens 42, in front of the inverted microscope 12, where the eyepiece 22 is arranged.

The main unit 50 includes an adapter holder 58, a Z-drive unit 60, and a needle position adjustment knob (not shown). The needle 64 having a tip portion 66, which should be driven, is attached to the adaptor 62. The adaptor 62 holding the needle 64 is attached to the adaptor holder 58. The Z-drive unit 60 moves the adapter holder 58 in a Z-direction to move the tip portion 66 in the Z-direction. The needle position adjustment knob moves the adapter holder 58 in the X-direction and the Y-direction to adjust an XY-position of the tip portion 66.

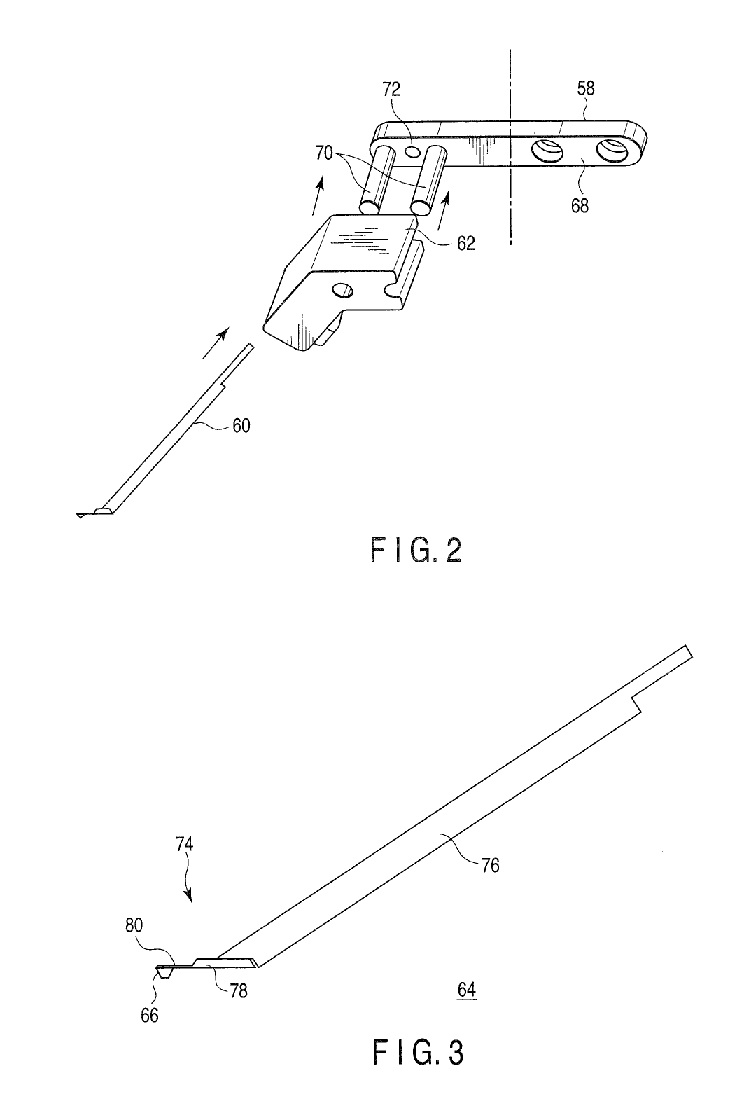

As shown in FIG. 2, the adapter holder 58 includes a Z-drive holder portion 68, an attachment member, and fitting portions 70. The Z-drive holder portion 68 is utilized to attach the adapter holder 58 to a non-illustrated linear movement mechanism of the Z-drive unit 60 through a non-illustrated XY-drive mechanism (the control box 56 drives the adapter holder 58 through this drive mechanism). The attachment member is configured to detachably attaches the adapter 62, and it is provided on a side of the adapter holder 58 opposite to the Z-drive holder portion 68 along a longitudinal direction. This attachment member is constituted of a magnet 72 if the adapter 62 is made of a metal or has a metal portion provided at a corresponding position, for example. It is to be noted that a right side of an alternate long and short dash line is a portion accommodated in the main unit 50 in FIG. 2. That is, the magnet 72 is provided at a position outside the main unit 50. The fitting portions 70 are arranged near this magnet 72 and fitted in holes or grooves provided in the adapter 62 for positioning of the adapter 62. These fitting portions 70 protrude toward the front surface side of the inverted microscope 12, whereby the adapter 62 can be attached from the front surface side by insertion.

It is to be noted that the magnet 72 and the fitting portions 70 may be likewise provided on a back surface side of the adapter holder 58 so that the adapter 62 can be attached even when the main unit 50 is attached to a left side of the condenser lens 42. Alternatively, the adapter holder 58 may be replaced by another, depending upon the position at the main unit 50 is secured.

As shown in FIG. 3, the needle 64 attached to the adapter 62 is composed of a cantilever chip 74 and a shaft 76. The tip portion 66 is formed at the cantilever chip 74. When the cantilever chip 74 is bonded to an end of the shaft 76, the shaft 76 holds the cantilever chip 74.

The cantilever chip 74 is manufactured by a silicon process and composed of a silicon base portion 78, a flexible lever portion 80, and the tip portion 66. The silicon base portion 78 is a portion that is bonded to another portion, i.e., the shaft 76. The lever portion 80 extends from the silicon base portion 78 and has, e.g., a thickness of 2.7 μm, a length of 240 μm, and an elastic constant of approximately 2 N/m. The tip portion 66 is formed at a free end of the lever portion 80 at an angle of approximately 90 degrees with respect to a longitudinal direction of the lever portion 80.

In the tip drive device 14 according to this embodiment, the needle 64 is inserted into/fixed in a non-illustrated hole formed in the adapter 62, and then the adapter 62 holding the needle 64 is attached to the main unit 50. The needle 64, which is basically a consumable supply and replaced frequently, can thus be replaced by a new one. Therefore, the tip drive device 14 can be repeatedly used without the risk of contamination.

Further, when a configuration that the elongated needle 64 is directly attached to the main unit 50 is adopted, workability is poor, and the tip portion 66 may hit on any position in the inverted microscope 12, e.g., the dish holder 16, and then, the tip portion 66 may be broken easily. In this embodiment, the needle 64 is attached to the adapter 62 removed from the main unit 50 and then the adapter 62 is attached from the front surface side of the main unit 50, thereby reducing the risk of such breakage.

It is to be noted that the adapter 62 is configured to hold the shaft 76 of the needle 64 obliquely downward at a predetermined angle when attached to the main unit 50, and the cantilever chip 74 is bonded to this shaft 76 to form a predetermined angle. Furthermore, as described above, the tip portion 66 is provided to extend in a direction crossing the longitudinal direction of the lever portion 80. Therefore, in a state where the main unit 50 has the adapter 62 attached thereto, the tip portion 66 is held at the free end of the lever portion 80 with an end thereof facing downward in a substantially vertical direction.

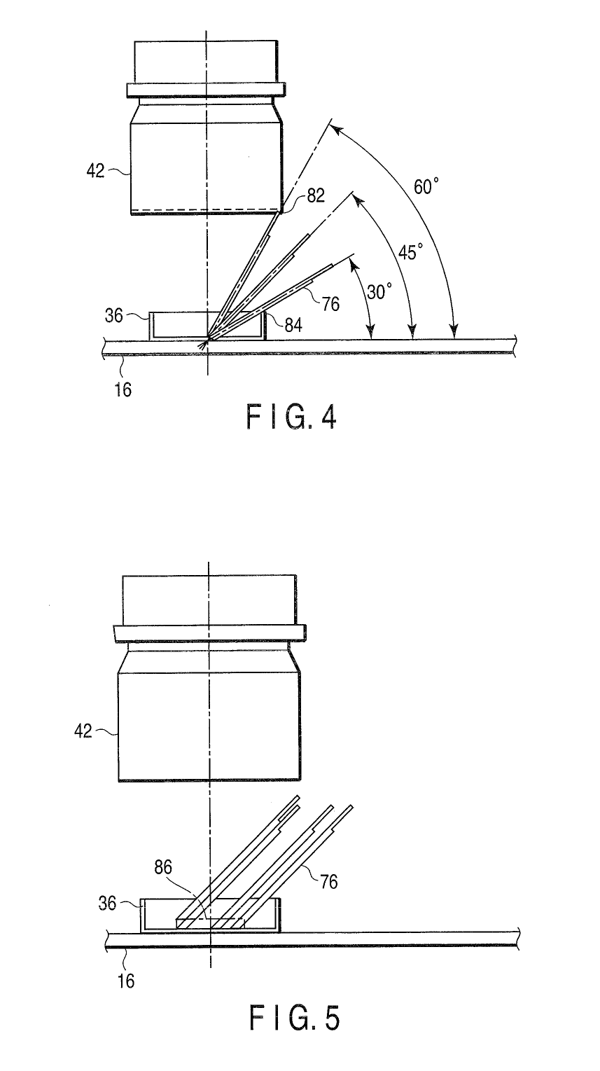

A fixed angle at which the adapter 62 holds the shaft 76 is determined as follows. That is, as indicated by reference number 82 in FIG. 4, when the shaft 76 is excessively raised, the shaft 76 interferes with the condenser lens 42. Assuming that a length of the needle 64 is, e.g., approximately 50 mm, when the shaft 76 is raised beyond 60 degrees, it interferes with the condenser lens 42. Contrary, when the shaft 76 is excessively inclined, the shaft 76 interferes with a circumferential wall portion of the dish 36 as indicated by reference number 84 in FIG. 4. In general, in case of a 35-mm glass bottom dish that is highly frequently used in cell culture, when the shaft 76 is inclined beyond 30 degrees, it interferes with the circumferential wall portion of the dish 36. Therefore, in this embodiment, the fixed angle at which the adapter 62 holds the shaft 76 is set to 45 degrees corresponding to an intermediate angle between 30 degrees and 60 degrees.

When the adapter 62 is set to hold the shaft 76 at the angle of 45 degrees, such a movable range 86 as indicated by an alternate long and short dash line in FIG. 5 is obtained, and the shaft 76 does not interfere with the condenser lens 42 or the circumferential wall portion of the dish 36 in regard to a glass surface (approximately φ14 mm) of the 35-mm glass bottom dish, thereby enabling an operation.

As described above, the fixed angle at which the adapter 62 holds the shaft 76 is determined to provide the sufficient movable range 86 to the needle 64 while considering the interference with the condenser lens 42 and the utilized dish 36. Further, a non-illustrated hole in which the needle 64 is inserted/fixed is formed in the adapter 62 with an angle enabling the shaft 76 to be held at this fixed angle.

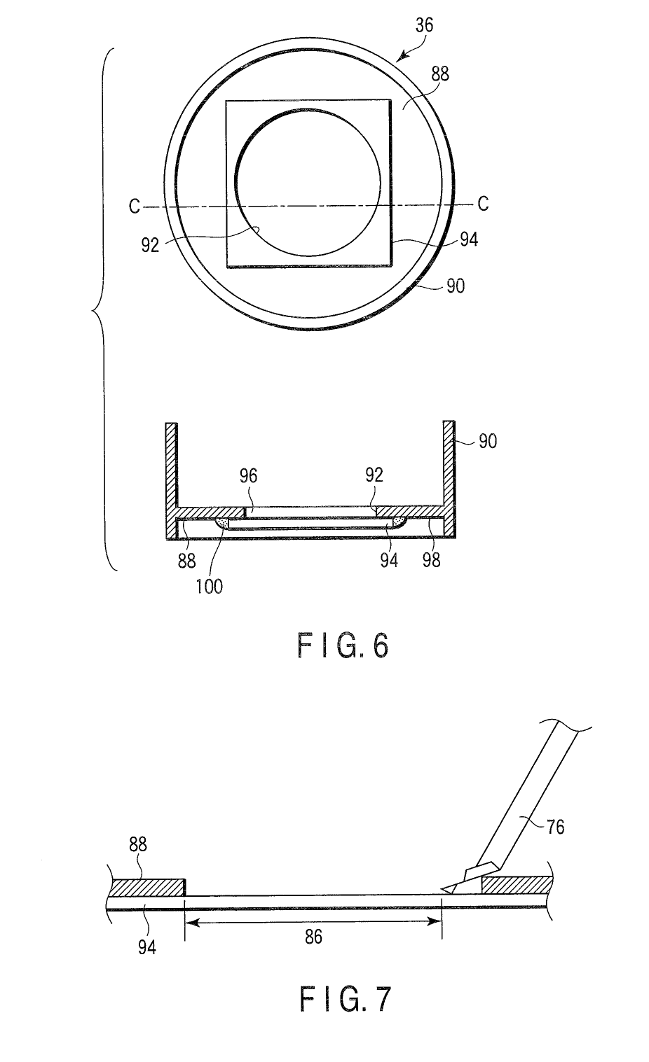

It is to be noted that the glass base dish utilized as the dish 36 is formed into a cylindrical shape with a bottom plate portion 88 and a circumferential side portion 90 consisting of a synthetic resin material such as polystyrene as shown in FIG. 6. One open hole 92 having a circular shape is formed at a central part of the bottom plate portion 88. A plate-like glass portion 94 that is sufficient to completely cover an aperture 96 of the open hole 92 is bonded from a lower surface 98 side of the bottom plate portion 88 by using an adhesive 100 consisting of a silicon-based adhesive or an acrylate-based adhesive. As a result, the entire aperture 96 of the open hole 92 is shielded by the plate-like glass portion 94. In this manner, the bottom plate portion 88 and the plate-like glass portion 94 constitute a bottom portion of the dish 36.

In the dish 36 having such a configuration, as shown in FIG. 7, the movable range 86 in which the needle 64 can be moved without the interference of the shaft 76 with the bottom plate portion 88 of the dish 36 is restricted to a range narrower than the aperture 96 of the open hole 92. This movable range 86 varies depending on, e.g., a diameter of the shaft 76 or a length of the cantilever chip 74. It is to be noted that the restriction based on the interference of this dish 36 with the bottom plate portion 88 can be of course avoided by providing the θ table 34 and driving the θ table 34 to rotate the dish 36.

As described above, in the cell manipulation observation apparatus 10 according to this embodiment, an observation range which is a range enabling observation by using the inverted microscope 12 is the entire aperture 96 of the open hole 92 of the dish 36 in which cells are cultured. On the other hand, a cell manipulation range which is a range enabling cell manipulation by using the tip drive device 14 is the movable range 86. Therefore, in the cell manipulation observation apparatus 10 according to this embodiment, the observation range and the cell manipulation range are different from each other.

On the other hand, although not shown in particular, the operation module 54 in the tip drive device 14 includes a Z-value set button. This Z-value set button is a button configured to issue a command for storing a desired position in the Z-direction. Even when the operation module 54 performs an operation of the Z-drive unit 60 to drive the adapter holder 58 in the Z-direction, the adapter holder 58 can be prevented from moving down below a position stored by this Z-value set button (direction of a cell in the dish 36). It is to be noted that this Z-value set button includes a latch mechanism, whereby a pressed state, i.e., an on-state is maintained until the button is again pressed when an operator performs a pressing operation, i.e., an on-operation. An off-state of the Z-value set button will be referred to as a “first mode” and the on-state of the same will be referred to as a “second mode” hereinafter.

Cell manipulation and an observation method using the cell manipulation observation apparatus 10 having the above-described configuration according to this embodiment will now be described hereinafter.

A description will be given as to an example where a substance is introduced into at least one of cells cultured in a culture fluid in the dish 36 and the at least one of cells having the substance introduced therein in this manner is observed.

An example of introducing a substance into a cell will be first explained.

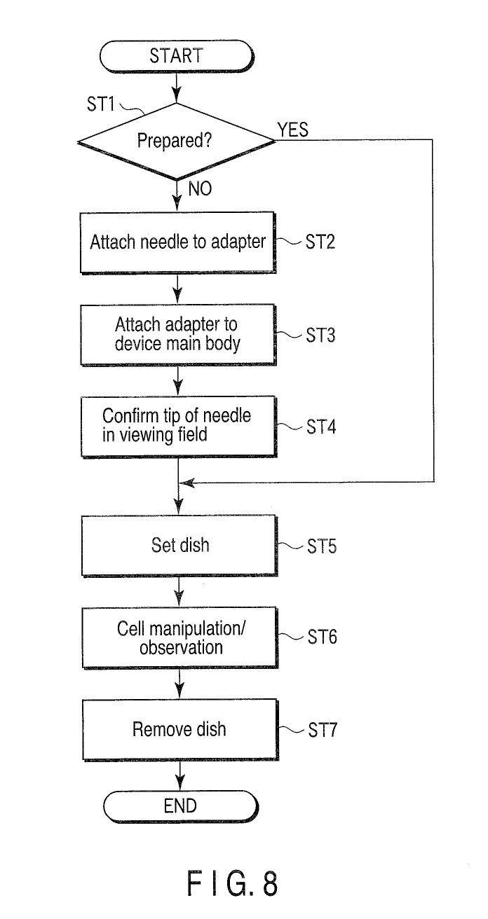

That is, as shown in FIG. 8, an operator of the cell manipulation observation apparatus 10 first confirms whether preparation of the tip drive device 14 required to perform cell manipulation has been finished (step ST1). Here, the preparation has not been finished, the needle 64 is inserted into and attached to the adapter 62 removed from the main unit 50 (step ST2). Additionally, the adapter 62 holding the needle 64 is attached to the adapter holder 58 of the main unit 50 attached to the condenser lens 42 through the microscope adapter 52 from the front surface side of the inverted microscope 12 (step ST3).

Then, the tip is positioned (step ST4). That is, the operator operates a non-illustrated needle position adjustment knob while observing tip portion 66 in the eyepiece lens 48 or the monitor 38 and sets a position of the tip portion 66 formed at the end of the needle 64 to a central position of the non-illustrated objective lens (central position in a viewing field) based on visual observation. This setting is performed without mounting the dish 36 on the dish holder 16.

When the tip is positioned in this manner or when it is confirmed that the tip drive device 14 has been prepared in step ST1, the operator then mounts the dish 36 onto the dish holder 16 (step ST5). It is to be noted that the dish 36 is set with a substance to be introduced being mixed in a culture fluid in order to introduce the substance into at least one of cells that is cultured in the culture fluid in the dish 36.

Further, when the XY-stage operation joystick 24 or the operation module 54 is operated to perform a cell manipulation/observation operation while confirming images displayed on the monitor 38, the substance is introduced into a desired cell(s) in the dish 36 (step ST6). This cell manipulation/observation operation is performed when the control computer 32 controls each unit in the inverted microscope 12 and the tip drive device 14 through the dish holder movement controller 26, the microscope controller 28, and the control box 56 in accordance with the operation effected by the operator, and particulars of this operation will be described later.

Further, when the introduction of the substance into the cell(s) is finished, the dish 36 is removed (step ST7).

Furthermore, when continuously introducing the substance into a cell(s) cultured in another dish 36, the above-described operation is repeated. In this case, since the preparation of the tip drive device 14 has been finished, the processing can advance from step ST1 to step ST5. Of course, to eliminate the risk of contamination, the processing may advance to step ST2 rather than step ST5 from step ST1 to replace the needle 64.

The dish 36 removed in step ST7 is subjected to cleaning to wash off the culture fluid in which the substance to be introduced is mixed, and it is then filled with a new culture fluid. As a result, the at least one of cells having the substance introduced therein is further cultured in the dish 36. Furthermore, after a predetermined period, observation is effected by using the cell manipulation observation apparatus 10.

In case of carrying out this observation, since the tip drive device 14 is not required, the processing advances to step ST5 from step ST1. Thus, the operator mounts the dish 36 in which the at least one of introduced cells to be observed is cultured onto the dish holder 16 that is moved in the horizontal direction by the microscope XY-stage 22.

Moreover, the operator operates the XY-stage operation joystick 24 or the operation module 54 to effect the cell manipulation/observation operation while confirming images displayed on the monitor 38, thereby observing the desired cell(s) in the dish 36 (step ST6).

Additionally, when the observation of the cell(s) is finished, the dish 36 is removed (step ST7).

The cell manipulation/observation operation in step ST6 will now be described.

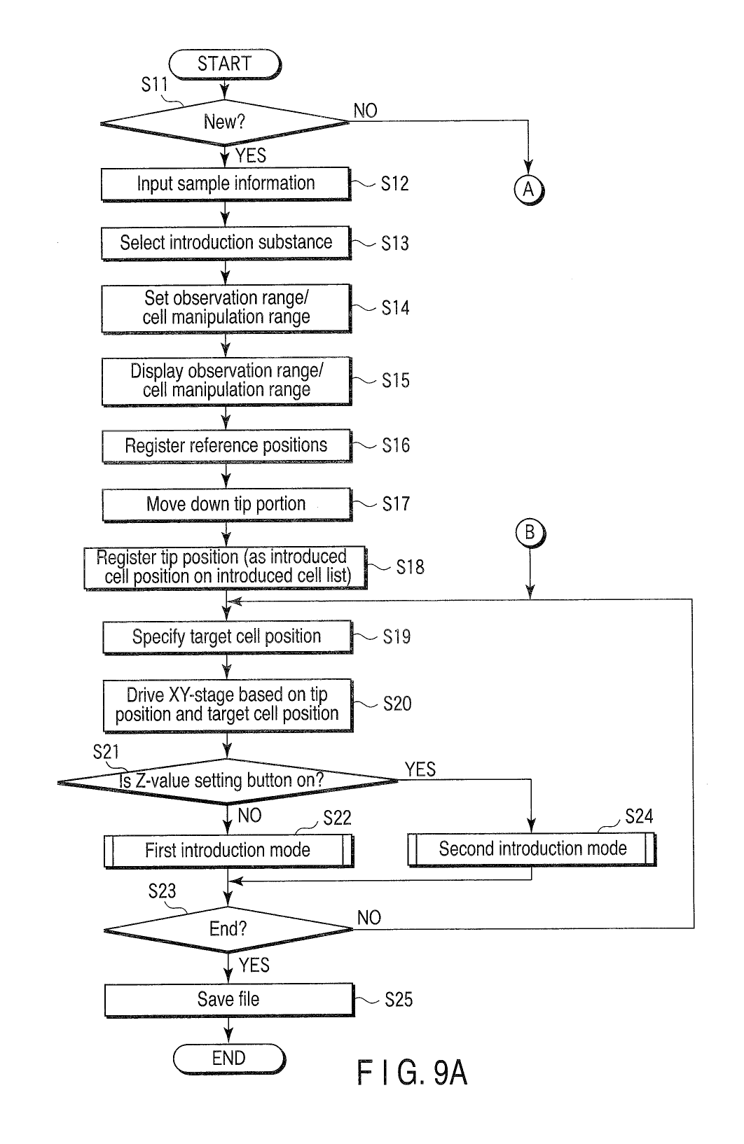

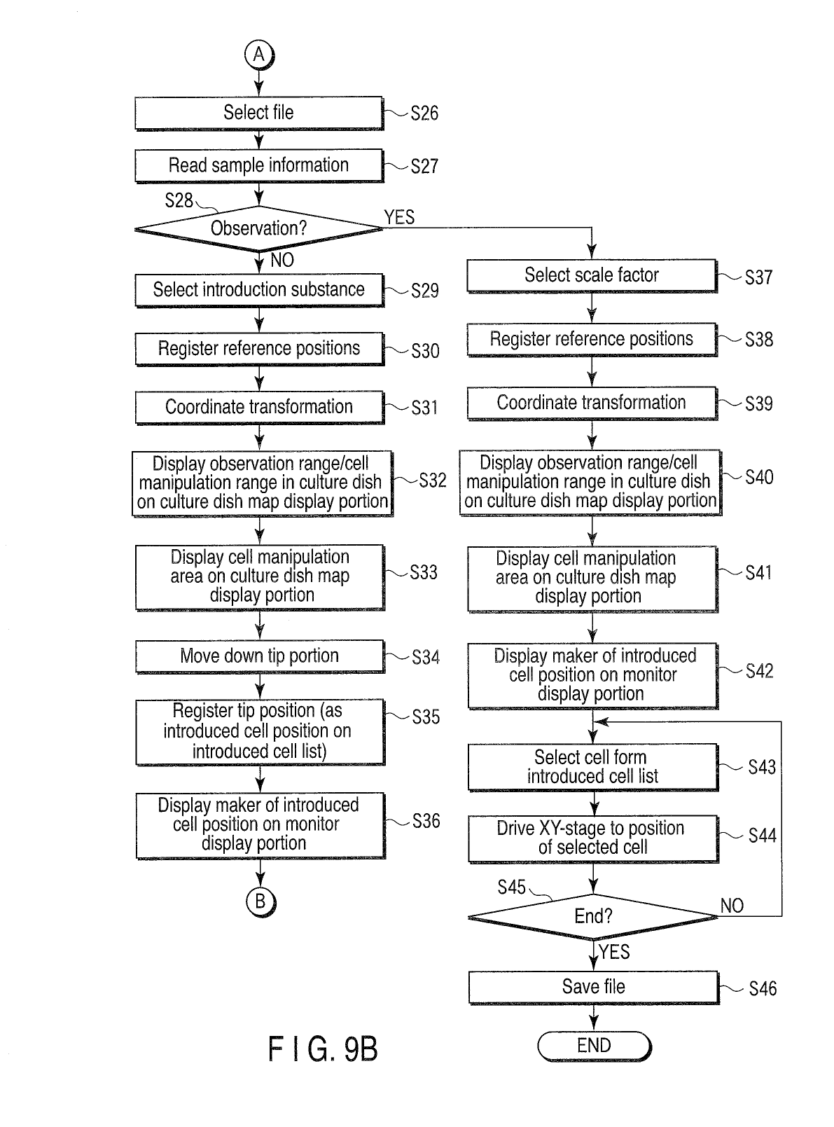

This cell manipulation/observation operation is carried out by allowing the control computer 32 to execute a control program that performs such operations as depicted in FIG. 9A and FIG. 9B.

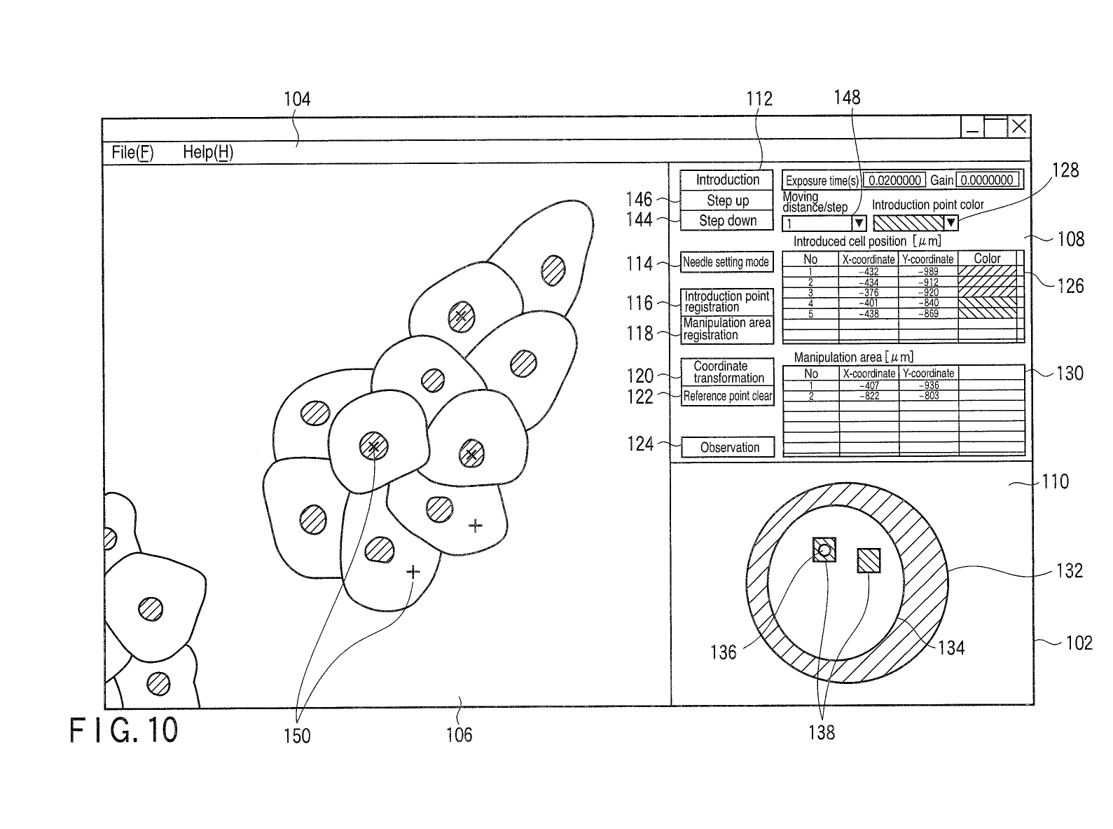

It is to be noted that, when the control program is activated, such an operation window 102 as depicted in FIG. 10 is displayed on the monitor 38. This operation window 102 has a menu bar 104, a monitor display portion 106, an operating portion 108, and an culture dish map display portion 110. The monitor display portion 106 is a region that displays an image that is acquired by the CCD camera 46 and processed by the image processing device 30. The operating portion 108 is a region that displays various operation buttons or lists. The culture dish map display portion 110 is a region that displays a portion in the dish 36 mounted on the dish holder 16 that is displayed by the monitor display portion 106.

The operating portion 108 includes an “introduction” button 112, a “needle setting mode” button 114, an “introduction point registration” button 116, a “manipulation area registration” button 118, a “coordinate transformation” button 120, a “reference point clear” button 122, an “observation” button 124, and others, as the operation buttons. The “introduction” button 112 is a button used for instructing to introduce a substance. The “needle setting mode” button 114 is a button used for instructing to register a position of the tip portion 66. The “introduction point registration” button 116 is a button used for instructing to register a position at which the substance is introduced. The “manipulation area registration” button 118 is a button used for instructing to register an area including a cell into which the substance is to be introduced. The “coordinate transformation” button 120 is a button used for instructing to register a reference point of the dish 36. The “reference point clear” button 122 is a button used for instructing to clear the registered reference point. The “observation” button 124 is a button used for instructing to perform observation.

Moreover, the operating portion 108 includes an introduced cell list 126. This introduced cell list 126 displays contents of an introduced cell list constituted in the memory unit 32A of the control computer 32. In this introduced cell list, introduced cell information of each cell is registered in accordance with a mouse click operation of the “introduction point registration” button 116. Here, the introduced cell information includes positional information (an XY-coordinate) and introduction substance information. The introduction substance information includes a display color in the monitor display portion 106, a substance name, a particle size, a concentration, observation conditions, and others. It is to be noted that the substance to be introduced may be introduced into a cell nucleus or a cytoplasm. For example, a gene, a pigment, a fluorescent reagent such as a quantum dot, an ion, a peptide, a protein, or a polysaccharide that can be muddled in the dish 36 can be adopted. It is preferable to previously register these substances to be introduced so that these substances can be selected in a pull-down list by using the introduction substance list 128. Displaying such an introduced cell list 126 enables recognizing introduction contents in accordance with each cell.

Additionally, the operating portion 108 includes a manipulation area list 130. This manipulation area list 130 shows contents of a manipulation area list constituted in the memory unit 32A of the control computer 32. In this manipulation area list, manipulation area information is registered in accordance with a mouse click operation of the “manipulation area registration” button 118. This manipulation area information includes positional information of an area including at least one of cells into which a substance is to be introduced or was introduced, e.g., an XY-coordinate of a predetermined point, e.g., an upper left part in a corresponding area. It is to be noted that a size of the manipulation area corresponds to a range displayed in the monitor display portion 106.

On the other hand, the culture dish map display portion 110 discriminably displays an observation range 132 and a cell manipulation range 134. Here, the observation range 132 is a view which corresponds to the entire aperture 96 of the open hole 92 of the dish 36 in which cells are cultured and is associated with an observable range of the inverted microscope 21. The cell manipulation range 134 is a range in which the cell manipulation can be performed by the tip drive device 14 in the observation range 132. Further, the culture dish map display portion 110 shows an area displayed in the monitor display portion 106, i.e., a currently observed position as a current position 136, and it also displays each area registered and displayed in the manipulation area list 130 as a cell manipulation area 138.

As described above, the culture dish map display portion 110 as diagram displaying unit displays the observation range 132 which is the observable range of the dish 36, and the cell manipulation range 134 which is a range where the cell manipulation is possible and the current position 136 which is a position currently observed in the monitor display portion 106, in the superimposing manner. When this display mode is used, the shaft 64 as the cell manipulation tool or the microscope XY-stage on which the dish 36 is mounted can be operated to prevent the current position from deviating the cell manipulation range 134, thereby eliminating the risk of accidentally striking the cell with the shaft 64. Further, since the monitor display portion 106 that displays an observation image of a current viewing field and the culture dish map display portion 110 are displayed on the same screen (operation window 102), a currently observed position can be easily recognized.

Referring to FIG. 9A, Furthermore, the control computer 32 first judges whether new file creation has been selected from a file menu displayed by, e.g., mouse clicking on a “file” item in the menu bar 104 in such an operation window 102 (step S11).

Here, when the new file creation has been selected, a non-illustrated sample information input window is additionally opened, input of sample information is accepted, and the input sample information is temporarily stored in the memory unit 32A (step S12). As this sample information, a date, a number, a cell, and others are included. A type of dish 36 and a type of needle 64 can be selected from the pull-down menu.

When the sample information has been input in this manner, a substance to be introduced is selected from the pull-down list of the introduction substance list 128 (step S13). At this time, a setting of a display color for identification and display in the introduced cell list 126 and the monitor display portion 106, a substance name, a size of a particle, a concentration, observation conditions, and others can be input. When observation conditions are input in advance, the observation conditions do not have to be set each time when observing a cell(s) having a substance introduced therein later. Such information of a substance to be introduced is also temporarily stored in the memory unit 32A.

Then, the microscope XY-stage 22 is driven by the dish holder movement controller 26 to move the dish holder 16 in the horizontal direction, an outer shape of the dish 36 is measured to set the observation range and the cell manipulation range, and these ranges are temporarily stored in the memory unit 32a (step S14). For example, based on the input type of dish 36, the observation range can be specified by measuring three points on an outer periphery if the dish 36 has a circular shape, or it can be specified by measuring two points of opposing corners if the dish 36 has a square shape. It is to be noted that this measurement can be automatically performed by detecting a boundary from an image acquired by the CCD camera 46 based on image processing and subjected to the image processing by the image processing device 30, or an operator may operate the XY-stage operation joystick 24 to specify the two points or the three points. That is, the dish holder movement controller 26, the control computer 32, the CCD camera 46, the image processing device 30, and others function as a measuring unit configured to measure a shape of the dish 36 mounted on the microscope XY-stage 22. Measuring the shape of the dish 36 in this manner enables easily setting the observation range.

Furthermore, the memory unit 32A of the control computer 32 previously stores a list of cell manipulation ranges associated with combinations of types of the dish 36 and types of the needle 64. Therefore, each cell manipulation range can be read from the memory unit 32A to be set in accordance with the type of dish 36 and the type of needle 64 that have been specified and temporarily stored.

Since the observation range and the cell manipulation range can be easily set in this manner, the set observation range and cell manipulation range and the current position displayed in the monitor display portion 106 are displayed as the observation range 132, the cell manipulation range 134, and the current position 136 in the culture dish map display portion 110, respectively (step S15).

Subsequently, registration of reference positions is accepted and temporarily stored in the memory unit 32A (step S16). In regard to this operation, when the operator sets the non-illustrated cursor on a desired position in the monitor display portion 106, performs a mouse click operation to specify the position, and operates the “coordinate transformation” button 120, the specified position is registered as the reference position in response to this button operation. The two reference positions can be registered. The registered reference positions are used for positioning (coordinate transformation or rotational driving on the θ table 34) when again setting and observing the temporarily removed dish 36.

Thereafter, the Z-drive unit 60 of the tip drive device 14 is activated through the control box 56 in accordance with an operation of the operation module 54 by the operator to move the tip portion 66 closer to the cell from above the cell (step S17). The operator moves down the tip portion 66 to a position at which the tip portion 66 and the cell in the dish 36 can be parfocality observed.

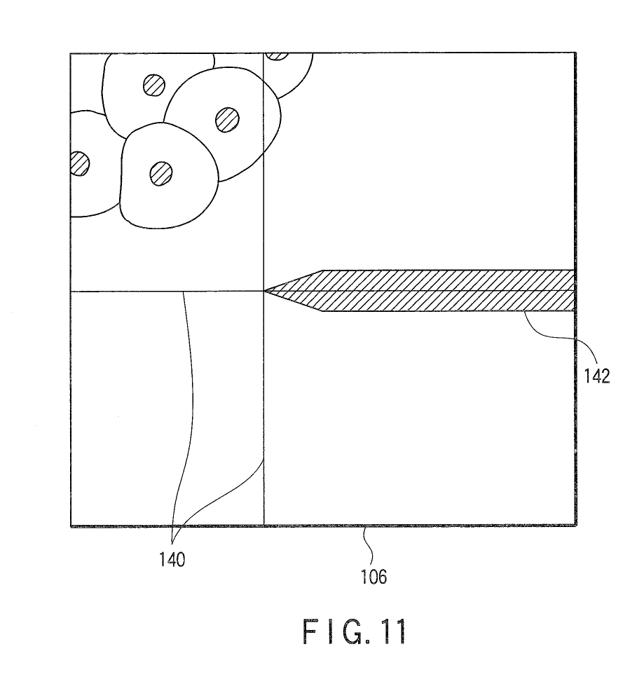

Moreover, a position of the tip portion 66 is registered in the memory unit 32A (step S18). With regard to this registration, when the operator operates the “needle setting mode” button 114, then sets the cursor 140 to an end portion of an image (cantilever chip image 142) of the cantilever chip 74 displayed in the monitor display portion 106, and performs a mouse click operation to specify a position of the tip portion 66, as shown in FIG. 11, the specified position is registered as a position of the tip portion 66 in accordance with the mouse click operation. In this manner, the position of the tip portion 66 can be registered. Additionally, when the same operation is again performed, the position can be rapidly corrected. As described above, the control computer 32 functions as a setting unit configured to set a predetermined cell manipulation position.

It is to be noted that the control computer 32 may automatically determine the position from a geometric configuration of the cantilever image 142 based on image processing in accordance with an operation of the “needle setting mode” button 114 in place of accepting the specification of the position by the operator. That is, the position of the tip portion 66 can be easily determined from the geometric configuration of the cantilever chip 74 without inserting the tip portion 66 into the cell.

As described above, since the shaft 76 of the tip drive device 14 is set up at a slant, accurately detecting the position of the tip portion 66 is difficult. Further, since displacement occurs due to deflection or a change occurs due to a damage when the tip portion 66 is inserted into the cell, the position of the tip portion 66 which is the predetermined cell manipulation position must be corrected each time. In this embodiment, the position of the tip portion 66 can be set without trouble by the specifying operation effected by the operator or by the automatic determination from the geometric configuration of the cantilever chip 74.

Then, specification of a position at which a substance is introduced is accepted (step S19). With regard to this acceptance, when the operator selects a cell into which a substance is to be introduced in the monitor display portion 106, places the cursor 140 on a position in the selected cell at which a substance is to be introduced, and performs a mouse click operation, the control computer 32 accepts this position as a specified position in accordance with this operation. In this manner, the control computer 32 also functions as a specifying unit configured to specify a position of the cell into which a substance is introduced of the cells in the dish holder 16. More specifically, the selection of the cell is carried out while viewing an image in the monitor display portion 106 as follows. That is, the dish 36 is moved to a predetermined cell manipulation position where cell introduction is effected by operating the XY-stage operation joystick 24 to drive the microscope XY-stage 22 through the dish holder movement controller 26. At this time, when the operator manipulates the XY-stage operation joystick 24 while confirming the display of the cell manipulation range 134 and the current position 136 in the culture dish map display portion 110, the shaft 76 can be prevented from striking the dish 36.

Furthermore, although not shown in the flowchart, the following specification method can be adopted. That is, an operation of first moving the dish 36 and operating the “manipulation area registration” button 118 to register a cell manipulation area in the manipulation area list provided in the memory unit 32A is repeated. Then, a desired cell manipulation area is selected in the manipulation area list 130 displaying contents of the manipulation area list in the memory unit 32A, and it is specified by a mouse double click operation, whereby a position of this manipulation area is displayed in the monitor display portion 106 as the cell manipulation area 138. Further, a cell into which a substance is to be introduced is selected from the displayed cell manipulation area 138.

Then, the control computer 32 calculates a relative position of a position of the tip portion 66 and the specified position and drives the microscope XY-stage 22 through the dish holder movement controller 26 to move the dish 36 to a position at which the specified position overlaps the position of the tip portion 66 (step S20). In this manner, the control computer 32 and the dish holder movement controller 26 function as an introducing position arrangement unit configured to drive the microscope XY-stage 22 to arrange the specified position at the predetermined cell manipulation position.

A subsequent operation differs depending on whether a Z-value has been set.

In the first tip driving, since the Z-value has not been set yet (step S21), a first mode cell introducing operation is performed (step S22).

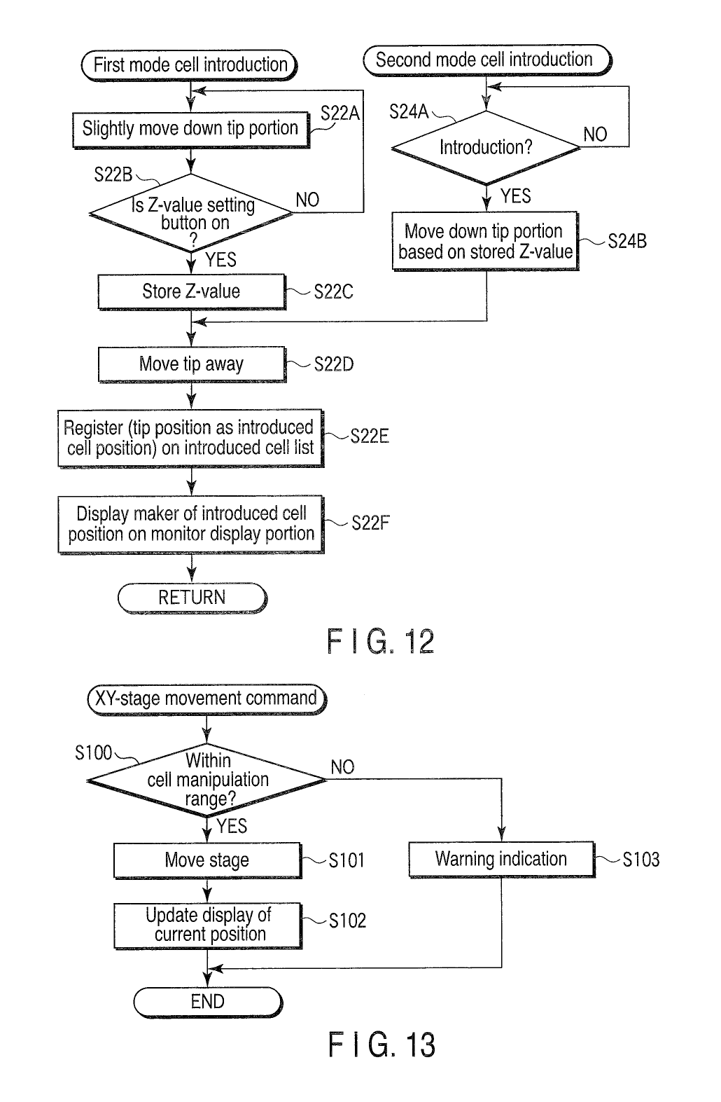

In this first mode cell introducing operation, as shown in FIG. 12, the tip portion 66 is moved down by a small distance at a time in the Z-direction to determine an optimum position in the Z-direction (step S22A). This operation is achieved when the operator operates the operation module 54 while observing images in the monitor display portion 106 to confirm “distortion of the cell” or “deflection of the lever portion 80” and the control computer 32 drives the Z-drive unit 60 through the control box 56 in accordance with this operation. At this time, the operation module 54 is operated while appropriately switching its sensitivity by using three levels, i.e., large, medium, and small of a non-illustrated distance setting dial provided in the operation module 54. Furthermore, as shown in FIG. 10, a “step down” button 144 and a “step up” button 146 and a step amount setting portion 148 may be provided in the operating portion 108, and a driving direction and a driving amount of the Z-drive unit 60 may be specified by performing mouse click operations with respect to these members in place of operating the operation module 54.

Moreover, the tip portion 66 is moved down to get closer to the bottom surface of the dish 36, and it comes into contact with a cell in the dish 36 in midstream of lowering the end of the tip portion 66. Here, when the tip portion 66 is further moved down, the end of the tip portion 66 will pass through the cell membrane and penetrates the cell nucleus, forming a scar or hole in the membrane and nucleus. The substance dispersed in the dish 36 therefore flows into the cell through the formed scar or hole. The substance may flow into the cell, without forming a scar or hole, depending on the size of particles to introduce, if the channel coupled to a stretch receptor of the like is opened when the tip portion 66 deforms the cell, applying a physical stimulus to the cell. Thus, the substance is introduced. At this time, the operator pushes the non-illustrated Z-value set button of the operation module 54. In response to the button pushing (step S22B), the control computer 32 stores in the memory unit 32A a position of the adapter holder 58 detected at this moment by a non-illustrated position detector as a Z-value indicative of an optimum position (step S22C).

Then, the tip portion 66 is raised by moving up the needle 64 in accordance with the operation of the operation module 54 (step S22D).

It is to be noted that, after the tip portion 66 is moved up to remove the tip portion 66 from the cell, when a given fixed time passes, the cell membrane is restored by self-reparation and the cell enters a state that the substance has been taken into the cell.

As described above, the tip drive device 14, the control computer 32, the operation module 54, and the control box 56 function as a cell manipulation unit configured to make a hole in the cell membrane of the cell that has been moved to the predetermined cell manipulation position and thereby introducing the introduction substance mixed in the culture fluid in the dish 36 into the cell.

After the substance has been introduced into the cell in this manner, the operator uses the mouse to click the “introduction point registration” button 116. In response to this operation, the control computer 32 registers a coordinate value (introducing position information) of the microscope XY-stage 22 associated with the position of the tip portion 66 and introduction substance information as information of an introduction point No. 1 in the introduced cell list provided in the memory unit 32A (step S22E). The registration contents are displayed as the introduced cell list 126 in the operating portion 108. That is, the introduced cell list 126 functions as a displaying unit configured to display the introduced cell information including cell positional information indicative of the specified position (introducing position information) and introduction substance information indicative of the introduced substance. Since the cell positional information and the introduction substance information are displayed in this manner, the cell and the substance introduced in this cell can be readily recognized. Additionally, the memory unit 32A functions as a storing unit configured to store the introduced cell information in cooperation with the operation of introducing the introduction substance into the cell. Since the introducing position information and the introduction substance information are automatically registered in cooperation with the substance introducing operation, information of the cell to which introduction has been tried is assuredly recorded, thereby avoiding a situation that the operator forgets registration.

Further, the monitor display portion 106 displays the positional information of the introduction point No. 1 as a marker 150 in a specified color (step S22F). This marker 150 is constantly displayed at a position of the introduction point No. 1 with movement of the microscope XY-stage 22. That is, when the position of the tip portion 66 is registered in advance, the tip portion 66 and the introduced cell position can be relatively positioned, and hence the cell into which the substance has been introduced can be determined, thereby accurately displaying the marker 150 indicative of a position in the cell to which the substance has been introduced.

Then, the substance is repeatedly introduced into each of desired cells.

That is, a judgment is made upon whether a predetermined end command, e.g., selecting end from the displayed file menu by using the mouse to click the “file” item in the menu bar 104 has been issued, (step S23), and the processing returns to step S19 to repeat the above-described operations if such an end command has not been issued.

In this case, in the second tip driving, since the Z-value has been set in the memory unit 32A (step S21), the second mode cell introducing operation is performed (step S24).

In this case, since the Z-value has been set in the memory unit 32A, after an XY-horizontal position is positioned, the operator can move the tip portion 66 to an optimum position by just effecting an operation of sufficiently lowering the tip portion 66 without concern for an excessive operation performed by the operation module 54. That is, as shown in FIG. 12, in accordance with the operation of the “introduction” button 112 by the operator (step S24A), the control computer 32 drives the Z-drive unit 60 through the control box 56 to move down the tip portion 66, compares a position of the adapter holder 58 detected by the non-illustrated position detector with the Z-value set in the memory unit 32A, and moves down the tip portion 66 until the adapter holder 58 (tip portion 66) reaches a position of the Z-value (step S24B).

Subsequently, the processing advances to step S22D, the needle 64 is moved up in accordance with the operation of the operation module 54, thereby raising the tip portion 66. When the substance is introduced into the cell in this manner, a coordinate value of the microscope XY-stage 22 associated with a position of the tip portion 66 and the introduction substance information are automatically registered as information of an introduction point No. n in the introduced cell list provided in the memory unit 32A, and registered contents are displayed as the introduced cell list 126 in the operating portion 108 (step S22E). Of course, like the first tip driving, these pieces of information may be registered in the introduced cell list in accordance with a mouse click operation performed with respect to the “introduction point registration” button 116. Further, the monitor display portion 106 displays positional information of the introduction point No. n in a specified color as the marker 150 (step S22F). This marker 150 is constantly displayed at a position of the introduction point No. n with movement of the microscope XY-stage 22.

Furthermore, an area observed at this moment in cooperation with the operation of introducing the introduction substance into this cell is stored in the manipulation area list in the memory unit 32A as the storing unit. As a result, the area where the cell manipulation has been carried out can be assuredly recorded. Moreover, when the control computer 32 superimposes a position of the cell manipulation area as the cell manipulation area 138 on the observation range 132 to be displayed in accordance with the stored cell manipulation area, the operator can recognize the area where the cell manipulation has been carried out.

When the instruction of the substance into each desired cell in the dish 36 is completed as described above, the operator issues a predetermined end command. Therefore, if such an end command is issued (step S23), the control computer 32 stores in the memory unit 32A information such as sample information, the introduction substance, the observation range, the cell manipulation range, the reference positions, the manipulation area list, the introduced cell list, and others temporarily stored in the memory unit 32A as a file under desired file name (step S25). Further, the temporarily stored information, e.g., the sample information, the introduction substance, the observation range, the cell manipulation range, the reference positions, the manipulation area list, the introduced cell list, and others are deleted from the memory unit 32A.

On the other hand, another substance may be further introduced with respect to the dish 36 where the given substance has been introduced as described above. In such a case, the culture fluid having the introduction substance mixed therein is washed off from the dish 36 temporarily removed in step ST7, and it is replaced by a culture fluid having a next introduction substance mixed therein. Then, this dish 36 is again mounted on the dish holder 16 in step ST5.

Furthermore, the operator carries out an operation of reading a file saved in step S25 from the file menu that is displayed when the mouse is utilized to click the “file” item in the menu bar 104 in the operation window 102.

That is, when new file creation is not selected from the file menu (step S11), the control computer 32 accepts selection of a file stored in the memory unit 32A (step S26), as shown in FIG. 9B. Moreover, information such as the sample information, the introduction substance, the observation range, the cell manipulation range, the reference positions, the manipulation area list, the introduced cell list, and others is read from the selected and accepted file to be temporarily stored in the memory unit 32A (step S27).

Subsequently, whether the mouse has been utilized to click the “observation” button 124 in the operating portion 108 is judged (step S28). If the “observation” button 124 has not been operated, an introduction substance is selected from the pull-down list of the introduction substance list 128 like step S13 (step S29).

Then, registration of each reference position is accepted, and the registered reference positions are temporarily stored in the memory unit 32A (step S30). In regard to this operation, when the operator sets the cursor to the same position as that specified in the monitor display portion 106 in step S16, specifies the position by a mouse click operation, and operates the “coordinate transformation” button 120, the control computer 32 registers the specified position as a current reference position in accordance with this button operation. The two reference positions are registered like step S16. Additionally, based on a difference between a coordinate value of the each registered current reference position and a coordinate value of each previous registered reference position temporarily stored in the memory unit 32A, each coordinate value in the introduced cell list and the manipulation area list temporarily stored in the memory unit 32A is converted into a coordinate value associated with a mounting state of the dish 36, and this value is temporarily stored in the memory unit 32A (step S31). It is to be noted that, in place of performing the coordinate value transformation to effect positioning in this manner, the θ table 34 may be rotated through the dish holder movement controller 26 and/or the microscope XY-stage 22 may be driven to move the dish 36 so that the coordinate values of the previous registered reference values can be met, thereby effecting the positioning. When the positioning is carried out based on the coordinate transformation or the movement of the dish 36 in this manner, a position(s) of the cell(s) into which the substance has been introduced can be reproduced after rearrangement of the dish 36.

Then, in accordance with the information of the observation range and the manipulation range temporarily stored in the memory unit 32A, the observation range 132 and the cell manipulation range 134 are displayed in the culture dish map display portion 110, and a current position displayed in the monitor display portion 106 is displayed as the current position 136 (step S32). Further, in accordance with the manipulation area list temporarily stored in the memory unit 32A, the cell manipulation area 138 subjected to the introduction is displayed in the culture dish map display portion 110 (step S33).

As described above, the control computer 32 and/or the dish holder movement controller 26 function as a reproducing unit configured to reproduce an observation state of the cell at the time of manipulating the corresponding cell based on the introduced cell information when the dish holder 16 is again arranged on the microscope XY-stage 22.

Thereafter, the tip portion 66 is moved closer to the cell from above the cell like step S17 (step S34), and a position of the tip portion 66 is registered in the memory unit 32A like step S18 (step S35). Furthermore, at this time, in accordance with the introduced cell list temporarily stored in the memory unit 32A, positional information of a previous introduction point(s) where the introduction has been effected is displayed in a specified color(s) as the marker(s) 150 on an image of the cells acquired by the CCD camera 46 and displayed in the monitor display portion 106 (step S36). Each marker 150 is constantly displayed at the position of the introduction point with movement of the dish 36 based on horizontal movement of the dish holder 16 by the microscope XY-stage 22.

Then, the processing advances to step S19 to execute the above-described operations.

In this manner, the plurality of types of introduction substances can be introduced. FIG. 10 shows a display example of the monitor display portion 106 when two types of introduction substances are introduced. In this case, the markers 150 are displayed in a different color depending on each introduction substance. It is to be noted that FIG. 10 shows a difference in color by a difference in shape of the markers 150, but actually the markers 150 have the same shape and colors alone are different. Of course, the introduction substances may be discriminable displayed based on the shapes of the markers 150 rather than the colors as shown in this drawing. In this case, an item “color” in the introduced cell list 126 changes to an item “marker shape” indicative of a marker shape, and the introduced cell list 128 also changes to a list of marker shapes rather than colors.

A cell manipulation/observation operation when carrying out observation will now be explained.

When carrying out the observation, the mouse is utilized to click the “observation” button 124 in the operating portion 108, and the observation is determined in step S28. Further, in this case, an observation scale factor is set through the microscope controller 28 in accordance with selection of an observation scale factor by the operator (step S37). That is, a fixed scale factor can be used in regard to the substance introduction, but performing the observation at various scale factors can be expected in regard to the observation, and hence an observation scale factor meeting a request from the operator is set.

Moreover, like step S30, registration of reference positions are accepted, and this registered positions are temporarily stored in the memory unit 32A (step S38). Additionally, based on a difference between a coordinate value of the each registered current reference position and a coordinate value of each registered reference position temporarily stored in the memory unit 32A, each coordinate value in the introduced cell list and the manipulation area list temporarily stored in the memory unit 32A is transformed into a coordinate value associated with a current mounting state of the dish 36, and this value is temporarily stored in the memory unit 32A (step S39). Alternatively, the dish 36 may be moved to meet the coordinate values of the registered reference positions by rotating the θ table 34 through the dish holder movement controller 26 and/or driving the microscope XY-stage 22.

Subsequently, like step S32, the observation range 132, the cell manipulation range 134, and the current position 136 are displayed in the culture dish map display portion 110 (step S40). Additionally, like step S33, the cell manipulation area 138 which has been subjected to introduction is displayed in the culture dish map display portion 110 (step S41). Further, like step S36, positional information of each introduction point where the introduction has been carried out is displayed in a specified color as the marker 150 in the monitor display portion 106 (step S42). Each marker 150 is constantly displayed at a position of the introduction point with movement of the dish 36 based on driving of the microscope XY-stage 22.

Thereafter, when the operator selects a desired introduced cell from the introduced cell list 126 in the operating portion 108 and performs a double click operation of the mouse, the cell as an observation target is selected (step S43). In accordance with this selection, the control computer 32 drives the microscope XY-stage 22 through the dish holder movement controller 26 to move the dish 36 to a position of the selected cell, thereby displaying the selected introduced cell in the monitor display portion 106 (step S44). Furthermore, based on information of observation conditions read from the selected file and temporarily stored in the memory unit 32A, the inverted microscope 12 is set through the microscope controller 28, and the observation is started. When the observation conditions are previously registered in this manner, automatic observation can be carried out. It is to be noted that the observation conditions can be appropriately changed at the time of this observation. Moreover, just selecting a cell from the introduced cell list 126 enables moving the dish 36 to a position of the introduced cell, thereby rapidly advancing the observation. Of course, the same operation can be carried out by not only selection from the introduced cell list 126 but also selection from the manipulation area list 130. When a plurality of observation conditions are provided with respect to the same area, these conditions are sequentially carried out, respectively.

Moreover, whether the operator has issued the predetermined end command is confirmed (step S45) and, if such an end command has not been issued, the processing returns to step S43 to observe another cell.

However, if the predetermined end command has been issued (step S45), information, e.g., the sample information, the introduction substance (including changed observation conditions), the observation range, the cell manipulation range, the reference positions, the manipulation area list, the introduced cell list, and others temporarily stored in the memory unit 32A is stored as a file in the memory unit 32A under a desired file name (step S46). Additionally, at this time, the temporarily stored information is deleted from the memory unit 32A.

### Second Embodiment

In a cell manipulation observation apparatus 10 according to a second embodiment of the present invention, when driving a microscope XY-stage 22 through a dish holder movement controller 26 in accordance with an operation of an XY-stage operation joystick 24, a mouse click operation in a monitor display portion 106, a mouse click operation for selecting an introduction point, or a mouse click operation for selecting a cell manipulation area, such an XY-stage movement command operation as shown in FIG. 13 is executed.

That is, first, whether a movement destination position is present within a cell manipulation range is judged (step S100) and, if it is present within the cell manipulation range, the microscope XY-stage 22 is driven through the dish holder movement controller 26 to horizontally move a dish holder 16, thereby moving a dish 36 (step S101). Additionally, display of a current position 136 in a culture dish map display portion 110 is updated (step S102).

On the other hand, if it is determined that the movement destination position is not present within the cell manipulation area in step S100, a warning indication is displayed on the monitor 38 without driving the microscope XY-stage 22 (step S103).

In the first embodiment, the operator confirms the cell manipulation range 134 and the current position 136 displayed in the culture dish map display portion 110 and instructs the microscope XY-stage 22 to move in such a manner that the shaft 76 does not strike the dish 36. On the other hand, in this second embodiment, a control computer 32 that functions as a drive controlling unit compares the cell manipulation range with the movement destination position before moving the microscope XY-stage 22 and avoids movement if the movement destination position is provided outside the cell manipulation range. Adopting such an operation enables reducing a burden on the operator and assuredly avoiding the risk that a shaft 64 as a cell manipulation tool accidently strikes a cell.

Although the present invention has been explained based on the embodiments, the present invention is not limited to the foregoing embodiments, and various modifications or applications can be of course carried out within the scope of the present invention.

For example, the operation window 102 shown in FIG. 10 is just an example, and the arrangement of the monitor display portion 106, the operating portion 108, and the culture dish map display portion 110 is not limited thereto. Further, the display of buttons or lists in the operating portion 108 is not limited either.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A cell manipulation observation apparatus which introduces a substance into a cell and observes the cell, comprising:
a stage configured to mount a culture dish and move the culture dish to horizontal;
an observation unit configured to acquire and display an image of the cells in the culture dish mounted on the stage;
a diagram display unit configured to display a diagram associated with an observation range which is a range of the culture dish observable by the observation unit and also display a cell manipulation range which is a cell manipulation enabled range and a position currently observed by the observation unit to be superimposed on the diagram associated with the observation range;
a specification unit configured to specify a position of a cell into which a substance is introduced of the cells in the culture dish mounted on the stage;
an introduction position arrangement unit configured to arrange the position specified by the specification unit to a predetermined cell manipulation position within a viewing field of the observation unit by driving the stage; and
a cell manipulation unit having a cell manipulation tool and configured to introduce an introduction substance mixed in a culture fluid in the culture dish into the cell by using the cell manipulation tool to perforate a cell membrane of the cell which has been moved to the predetermined cell manipulation position by the introduction position arrangement unit.

2. The apparatus according to claim 1, further comprising:
a memory unit configured to store a list of cell manipulation ranges associated with a type of the culture dish and a type of the cell manipulation unit; and
a type specification unit configured to specify the type of the culture dish and the type of the cell manipulation unit,
wherein the diagram display unit reads the cell manipulation range from the list stored in the memory unit in accordance with the types of the culture dish and the cell manipulation unit specified by the type specification unit and displays the read cell manipulation range.

3. The apparatus according to claim 2, further comprising a measurement unit configured to measure a shape of the culture dish mounted on the stage,
wherein the diagram display unit specifies an observation position on the stage based on the shape of the culture dish measured by the measurement unit to display the diagram.

4. The apparatus according to claim 1, wherein the display of the cell by the observation unit and the display of the diagram by the diagram display unit are carried out on the same screen.

5. The apparatus according to claim 1, further comprising a driving control unit configured to perform control of avoiding driving of the stage when the introduction position arrangement unit compares the position specified by the specification unit with the cell manipulation range and the specified position deviates from the cell manipulation range.

6. The apparatus according to claim 1, further comprising a memory unit configured to store as a cell manipulation area an area observed by the observation unit at the time in cooperation with manipulation of introducing an introduction substance into the cell by the cell manipulation unit,
wherein the diagram display unit further superimposes a diagram showing the cell manipulation area on a diagram associated with the observation range in accordance with the cell manipulation area stored in the memory unit and displays the superimposed diagrams.
